(19) United States
(12) Patent Application Publication
(10) Pub. No.: US 2007/0045143 A1
(43) Pub. Date: Mar. 1, 2007

(54) Package of flexible absorbent articles

(76) Inventors: Jane L. Clough, Neenah, WI (US); Kellie M. Goodrich, Appleton, WI (US); Dean M. Wydeven, Appleton, WI (US)

Correspondence Address: KIMBERLY-CLARK WORLDWIDE, INC., 401 NORTH LAKE STREET, NEENAH, WI, 54956, US

(21) Appl. No.: 11/212,951

(22) Filed: Aug. 26, 2005

(51) Int. Cl. A61B 19/02

(52) U.S. Cl. 206/440

(57) ABSTRACT

A parcel of flat absorbent articles such as diapers placed into a stack and disposed into an encasement. When the encasement is in a closed state, the stack is folded. When the encasement is in at least a partially open state, one of the absorbent articles may be dispensed from the encasement.

### BACKGROUND OF THE INVENTION

The present invention relates generally to a package or parcel of disposable absorbent articles and related accessories. More particularly, the present invention relates to a clam-shell style package of diapers, incontinent adult care products, catamenial products, and related accessories.

Diapers are generally a necessity for infants, toddlers and incontinent adults. Individuals typically need to carry extra disposable diapers and changing accessories such as wipes, powder and the like. Storage, carriage, and preparation of a clean diaper for application are often inconvenient. Typically, a bundle of diapers are individually bi-folded, compressed and packed into a flexible plastic bag or the like. When traveling, users take a few folded diapers out of the bag and may place them in a purse or bag for later use. However, after being removed from their original package, bi-fold diapers often unfold or possibly expand to consume an even larger storage space.

In addition, it is often the case that it is not convenient to prepare a bi-folded, multi-folded and or individually packed diaper for use. The diaper needs to be unfolded prior to use. For instance, the caretaker of an infant or child needs to unfold the diaper and position it so that the inner surface or body-facing surface of the diaper is face up on a table, floor or other substantially horizontal surface. It may also be necessary to unfold ears and tabs so that they extend outwardly from the absorbent portion of the diaper. During a diaper change when one may need to hurry or struggle with an uncooperative child, the extra steps necessary to prepare the diaper for use are not desirable.

Further, it may not be sufficient to simply change a soiled diaper and replace it with a clean diaper. For instance, cleaning of the individual who wore the diaper may need to be performed with diaper accessories, such as moist wipes. One may also want to apply ointment or powder to the individual wearing the diaper to treat or prevent incidents of diaper rash or other skin conditions. Due to these considerations, caretakers of individuals requiring diapers often carry diaper changing accessories in a dedicated container so that all the accessories are in one location. While a diaper bag or the like provides a dedicated container, one must typically forage through the bag to find the needed accessory. This can leave the person changing a diaper on an uncooperative child with no free hands to control the child.

Thus, there remains a need for a convenient package of absorbent articles that does not require the caretaker to prepare the diaper by unfolding tabs and/or laying the article out in an open state. There further remains a need for a package of diapers that includes easily accessible diaper changing accessories.

### SUMMARY OF THE INVENTION

A parcel of disposable personal-care products designed to be worn on the lower torso. The parcel has a first product having a pair of first surfaces, namely, a first body-facing surface and a first garment-facing surface; and a first fold-line. Further included is a second product having a pair of second surfaces, namely, a second body-facing surface and a second garment-facing surface; and a second fold-line. A stack of the first product and the second product is formed wherein one of the pair of first surfaces is in contacting relationship with one of the pair of second surfaces. An encasement adapted to have an open condition and a closed condition has the stack disposed therein. When the encasement is in a closed condition, the first product is substantially folded at the first fold line and the second product is substantially folded at the second fold line. When the encasement is in an open condition, the first and second products are at least partially unfolded.

In another aspect, the invention is an encasement for selectively dispensing absorbent products for wear about the lower torso. The encasement has a first shell member, and a second shell member mateable with the first shell member to define an inner recess adapted to enclose a stack of absorbent articles. A stack holder is adapted to dispense the absorbent articles. A connector attaches to the first shell member and second shell member so that the encasement may be selectively opened and closed.

In yet another embodiment, a parcel is defined by a plurality of disposable diapers, wherein the plurality of disposable diapers comprises a stack of unfolded diapers. The encasement is adapted to have an open condition and a closed condition. A stack is disposed within the encasement so that when the encasement is in a closed condition, the stack is substantially folded, and when the encasement is in an open condition, the stack is at least partially unfolded.

### BRIEF DESCRIPTION OF THE DRAWINGS

FIG. 1. representatively illustrates a plan view of an exemplary absorbent article in a laid flat condition with the surface of the article that contacts the skin of the wearer facing the viewer;

FIG. 2 representatively illustrates a front perspective view of one embodiment of an encasement of the present invention, shown in a partially closed state;

FIG. 3 illustrates a front elevation view of the encasement shown in FIG. 2, the encasement in a fully closed state;

FIG. 4 illustrates a side elevation view of the encasement shown in FIG. 3;

FIG. 5 illustrates a phantom view of the encasement shown in FIG. 4, with an exemplary folded stack of absorbent articles disposed therein;

FIG. 6 illustrates a plan view of the encasement shown in FIG. 2 in a fully open state and containing the absorbent article of FIG. 1;

FIG. 7 illustrates an exploded front perspective view of the encasement and absorbent articles of FIG. 6;

FIG. 8 illustrates a top, perspective view another embodiment of the encasement of the present invention, shown in an open state.

FIG. 9 illustrates a side perspective view of yet another embodiment of the encasement of the present invention, shown in an open state with an exemplary stack of absorbent articles disposed therein.

FIG. 10 illustrates a front perspective view of yet another embodiment of the encasement of the present invention.

Corresponding reference numbers and characters indicate corresponding parts throughout the drawings.

### DETAILED DESCRIPTION

The invention will now be described in detail with reference to particular embodiments thereof. The embodiments are provided by way of explanation of the invention, and are not meant as a limitation of the invention. For example, features described or illustrated as part of one embodiment may be used with another embodiment to yield still a further embodiment. It is intended that the present invention include these and other modifications and variations as come within the scope and spirit of the invention.

The present invention provides a package of flexible absorbent articles arranged into a stack and stored within a clam-shell type encasement. The flexibility of the stack is provided though material selection, article design, and construction of each absorbent article within the stack. Relatively thin, flat absorbent articles may be placed into a stack that is folded over in its entirety for convenient storage and carriage. An encasement that surrounds the folded stack is provided by the present invention. This encasement surrounds the stack when in a closed state, and unfolds the stack to present an absorbent article for dispensing when in an open state.

The absorbent article of the present invention will be described in terms of a diaper adapted to be worn by infants about the lower torso. It is understood that the absorbent article of the present invention is equally applicable to other articles such as adult incontinent products, training pants, feminine care products, and the like.

Within the context of this specification, each term or phrase below includes the following meaning or meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

The terms “disposed on,” “disposed along,” or “disposed toward” and variations thereof are intended to mean that one element can be integral with another element, or that one element can be a separate structure bonded to or placed with or placed near another element.

“Elastic,” “elasticized,” “elasticity,” and “elastomeric” mean that property of a material or composite by virtue of which it tends to recover its original size and shape after removal of a force causing a deformation. Suitably, an elastic material or composite can be elongated by at least 25 percent (to 125 percent) of its relaxed length and will recover, upon release of the applied force, at least 40 percent of its elongation. Desirably an elastic material or composite be capable of being elongated by at least 100 percent (to 200 percent), more desirably by at least 150 percent (to 250 percent), of its relaxed length and recover, upon release of an applied force, at least 50 percent of its elongation.

“Hook and loop fasteners” refers to fastening means comprising a “hook” component and a complementary loop component. The term “hook” is used to designate a material having protruding or engaging elements. It should also be understood that the use of the term “hook” should be non-limiting in the sense that the engaging elements may comprise any shapes as are known in the art so long as they are adapted to engage a complementary fastener component or material characterized by loops or the like.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Stretchable” means that a material can be stretched, without breaking, by at least 25 percent (to 125 percent of its initial (unstretched) length) in at least one direction, suitably by at least 100 percent (to 200 percent of its initial length), desirably by at least 150 percent (to at least 250 percent of its initial length) and may or may not recover properties upon release of an applied force. Elastic materials and extensible materials are each stretchable materials.

“Superabsorbent material” refers to a water-swellable, water-insoluble organic or inorganic material capable, under the most favorable conditions, of absorbing at least about ten times its weight and, more desirably, at least about thirty times its weight in an aqueous solution containing about 0.9 weight percent sodium chloride.

These terms may be defined with additional language in the remaining portions of the specification.

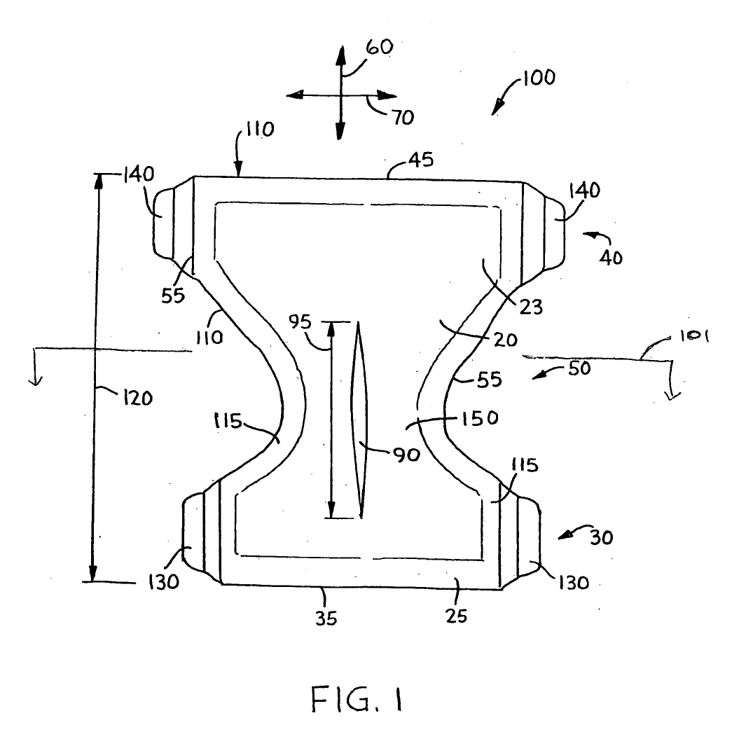

Many configurations of absorbent article may be used in conjunction with the encasement of the present invention. FIG. 1 representatively illustrates just one embodiment of an absorbent article that may possibly be used in the present invention; in particular, shown is a disposable absorbent article or garment in a flat, laid-out configuration, identified by reference number 100. However, the invention is not intended to be limited to this particular embodiment.

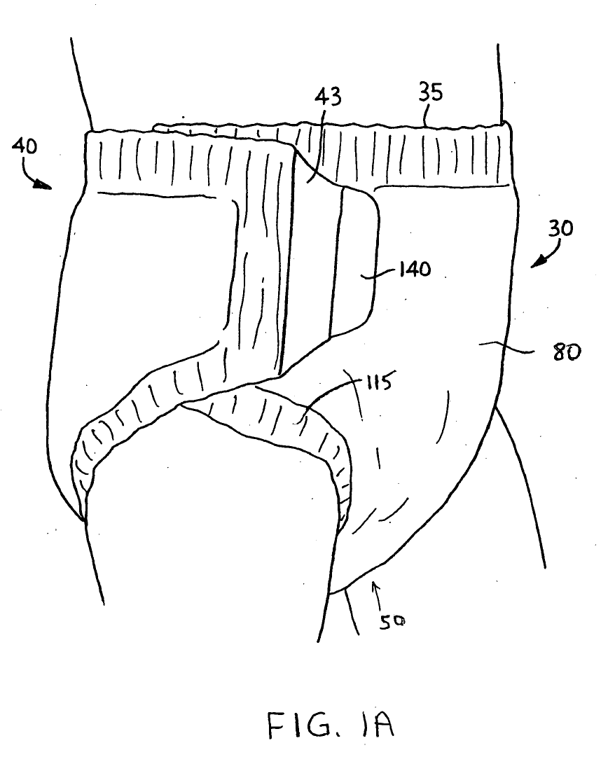

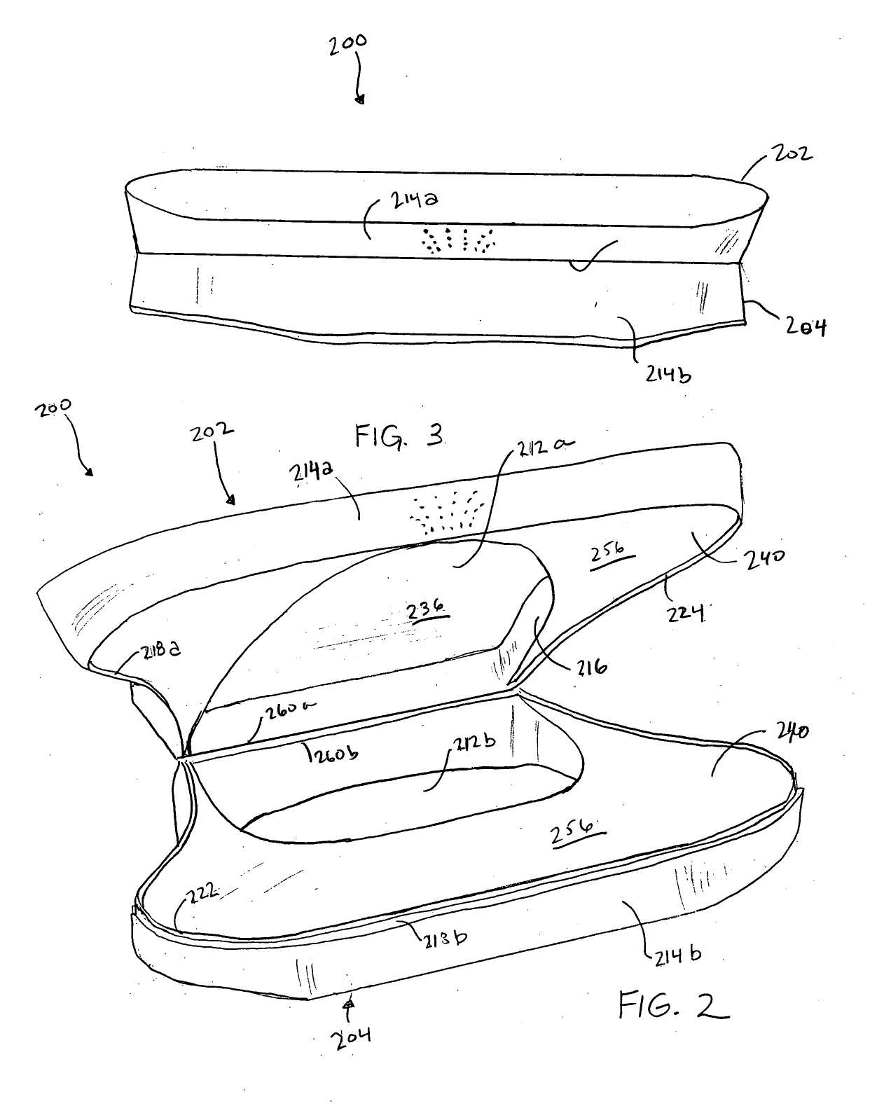

The disposable garment 100 may include an outer layer 80 (see FIG. 2) and an inner layer 20. The disposable garment 100 also has a pair of major surfaces designated as a body-facing surface found at inner layer 20, and a garment-facing surface found at outer-layer 80. The disposable garment 100 generally defines a front waist region 30, a back waist region 40 and a crotch region 50 in between the front waist region 30 and the back waist region 40. The disposable garment 100 has a longitudinal direction 60 aligned with the length 120 of the disposable garment 100 and a lateral direction 70 aligned with the width of the disposable garment 100. The lateral direction 70 is substantially perpendicular to the longitudinal direction 60. This particular example of an absorbent article is disclosed in commonly owned patent application, U.S. Ser. No. 10/750,402, incorporated herein by reference to the extent that it is not contradictory with the description provided herein.

The inner layer 20 may include an elastic material so that the inner layer 20 is elastically stretchable in the longitudinal direction 60, the lateral direction 70 or both the longitudinal direction 60 and the lateral direction 70. The outer layer 80 may be formed of a stretchable material such that the outer layer is extensible or elastic. Alternatively, as with the inner layer 20, when the outer layer 80 is formed of an elastic material, the outer layer 80 is capable of elastically stretching in the longitudinal direction 60, the lateral direction 70 or both the longitudinal direction 60 and the lateral direction 70.

Various materials may be provided for the inner layer 20 and the outer layer 80. Such materials may be non-extensible or extensible, and may suitably be elastic. Suitable nonwoven materials include nonwovens, elastic nonwoven materials, and laminates of nonwoven and elastic materials. The inner layer 20 and the outer layer 80 may also be formed from elastically stretchable or extensible film materials. The outer layer 80, may also be formed from such materials.

The inner layer 20, may present a bodyfacing surface 23 that is be compliant, soft feeling, and nonirritating to the wearer's skin. The inner layer 20 may define an opening 90 where the opening 90 may be located in an internal position to the perimeter 25 of the elastic inner layer 20. The inner layer perimeter 25 generally corresponds to the border defined by the elastic inner layer 20. The size of the inner layer 20 relative to the outer layer 80 may be considered in terms of surface area. For example, the inner layer 20 may have a smaller surface area than the outer layer 80.

Fasteners, such as fasteners 140 may be used to join the back waist region 40 with the front waist region 30 when the garment 100 is donned. In one embodiment, fasteners 140 may be provided near the longitudinal side edge 55 of the garment 100 and near where the inner layer perimeter 25 is bonded to the outer layer 80. Other locations are contemplated, depending on the design of the absorbent article. In addition, the type of fastener used is dependent on whether or not refastenability is desired, the stiffness of the overall garment, shape, and other considerations as is known in the art.

The garment 100 may be constructed so that there is no need to incorporate individual, highly-curved elastic components. Elimination of the need to incorporate separate elastic components results in a garment 100 that lies flat. This feature may be desirable when the garment 100 is in the form of an infant diaper. Diapers that lie flat may be easier to apply to infants and toddlers than diapers that want to curl closed. In addition, diapers that lie flat may be packed or stored differently because it may be easier to create a stack of unfolded garments 100.

The disposable garments 100 may include any form of fastener to maintain the garment on the wearer during use. For example, disposable garments 100 may include mechanical fasteners 130 and 140 for attaching the front waist region 30 and the back waist region 40 together. The material forming the mechanical fasteners 130 and 140 may be integral with the inner layer 20 and the outer layer 80, or the material may be provided on separate portions (e.g. front ear portion 33 and back ear portion 43, both shown in FIG. 2) that are attached to the longitudinal side edges 55 of the garments 100 in the waist regions. The mechanical fasteners 130 and 140 may include a variety of materials and surfaces known for mechanical engagement such as buttons, snaps, adhesive tape fasteners, cohesives, mushroom-and-loop fasteners, hook and loop fasteners, and the like.

The absorbent article 100 of the present invention may be of any thickness that allows the garment to fold. In particular, the garment may have a thickness of less than about 4 mm at the thickest portion of article. For a diaper-type product, it may be that the thickness portion of the diaper, typically the absorbent portion at or near the crotch portion, be between about 1 mm to 4 mm thick, or possibly between about 1 mm and 3 mm, or even between about 1 mm and 2 mm thick.

Thickness Test The thickness of an absorbent article such as garment 100 can be measured in accordance with the following procedure. All measurements are made on newly unpacked absorbent articles within one hour from start of specimen preparation to completion of test. Unless otherwise stated, all tests are performed at a relative humidity of 50%+/−2% and a temperature of 73 degree F. Each absorbent article should be removed from its package and if necessary, all elastics removed from the absorbent article to allow it to lie in a flat unwrinkled condition. The absorbent article must be handled carefully to avoid disturbing or changing the thickness.

A thickness tester, consisting of a digital indicator (Sony Model U30A, available from Sony Corporation, Tokyo, Japan) suitably attached to a comparator base (Starrett model 653G, available from L.S. Starrett Company, Athol, Mass.) is used. A 2.0 inch (5.08 cm) diameter circular platen made of brass is attached to the thickness indicator shaft which together with the shaft weight provides a pressure of 0.2 psi. The thickness indicator platen is first zeroed against the comparator base plate. The platen is then raised and the absorbent article is placed on the base plate, garment surface down. The absorbent article is positioned on the base plate so that when the platen is lowered it is in the region of the absorbent article for which the measurement is desired. The platen is then gently lowered onto the absorbent article. The absorbent article thickness is determined by reading the indicator 3.0 seconds after the platen comes in contact with the absorbent article.

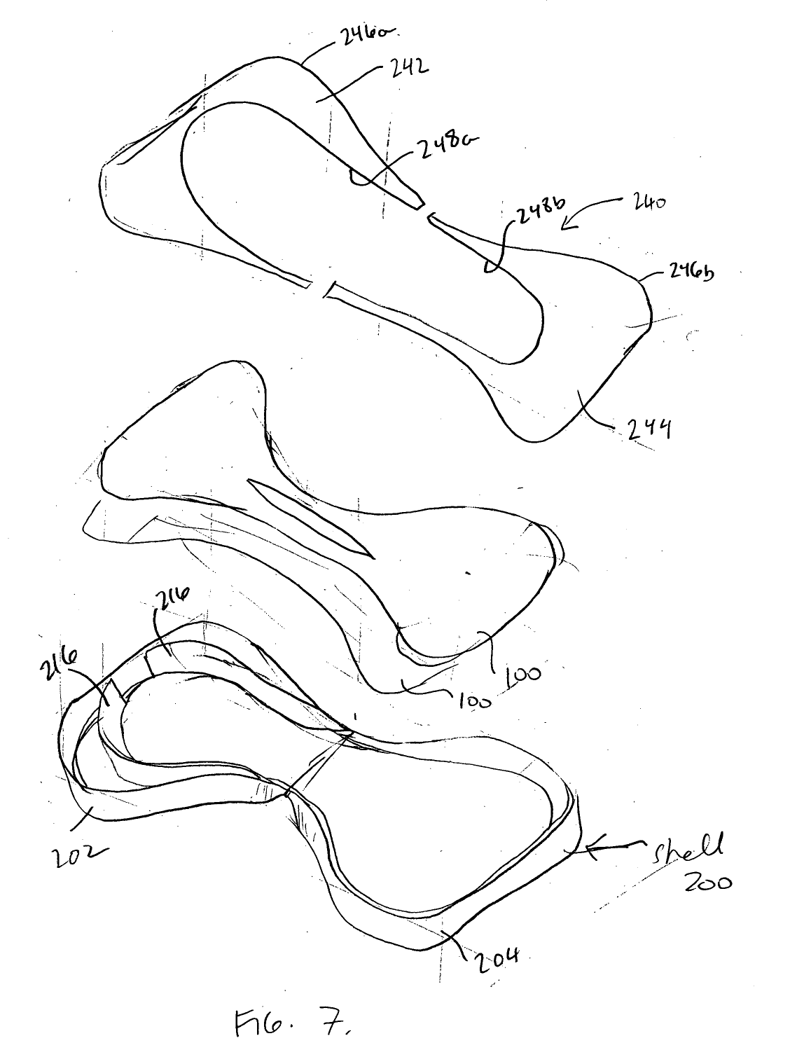

Absorbent articles 100 may be placed into a stack 192. Referring to FIG. 7, the stack may be created by placing one body- or garment-facing surface of one unfolded garment 100 into contact with the body- or garment-facing surface of another unfolded garment 100. If more than two garments are desired, this step may be repeated until several garments 100 are piled on top of one another. Each garment 100 defines an outer perimeter, which may be substantially identical for each garment 100. Likewise each garment 100 has a transverse axis in direction 70 and a longitudinal axis in direction 60, such directions shown in FIG. 1. When creating the stack 192, the transverse and longitudinal axes may be aligned so that the outer perimeters, if identical, are also in substantial alignment before the stack 192 is folded. Desirably, the contact between each of the absorbent articles is a full surface contact such as when the perimeters are aligned. When stack 192 is folded over, the innermost garment 100a will likely exhibit a relatively sharply defined fold or crease; whereas the outmost garment 100 may exhibit a less-sharply defined fold.

Referring now to FIG. 2, the packaging for the absorbent article, designated as encasement 200, defines a volume for receipt of stack 192 of personal care absorbent articles. In one embodiment, the absorbent articles are garments 100. The encasement 200 is not limited by its shape, materials, or configuration, so long as it is appropriate for retaining a stack 192. In one embodiment, the encasement 200 may be a relatively rigid body having a shape that generally conforms to the shape of the garments 100 intended to be placed in the encasement 200. Alternatively, encasement 200 may have a relatively flexible body that may or may not conform to the shape of the garments 100. The encasement 200 may have a generally nondescript appearance so that it gives no indication of the absorbent garments 100 carried therein, and/or may be relatively ornate or decorative. A vast number of encasement 200 configurations are within the scope and spirit of the invention.

In a first embodiment as depicted in FIGS. 2-7, the encasement 200 has a first shell member 202 and a second shell member 204 connected together by a hinge 206. Hinge 206 allows the encasement 200 to be selectively opened and closed. In this particular embodiment, the overall shape of encasement 200 is generally governed by the article it is designed to contain, and in particular, the garment 100 of FIG. 1. Even more specifically, the encasement 200 is designed to hold a stack of garment 100 as shown in FIGS. 5-7.

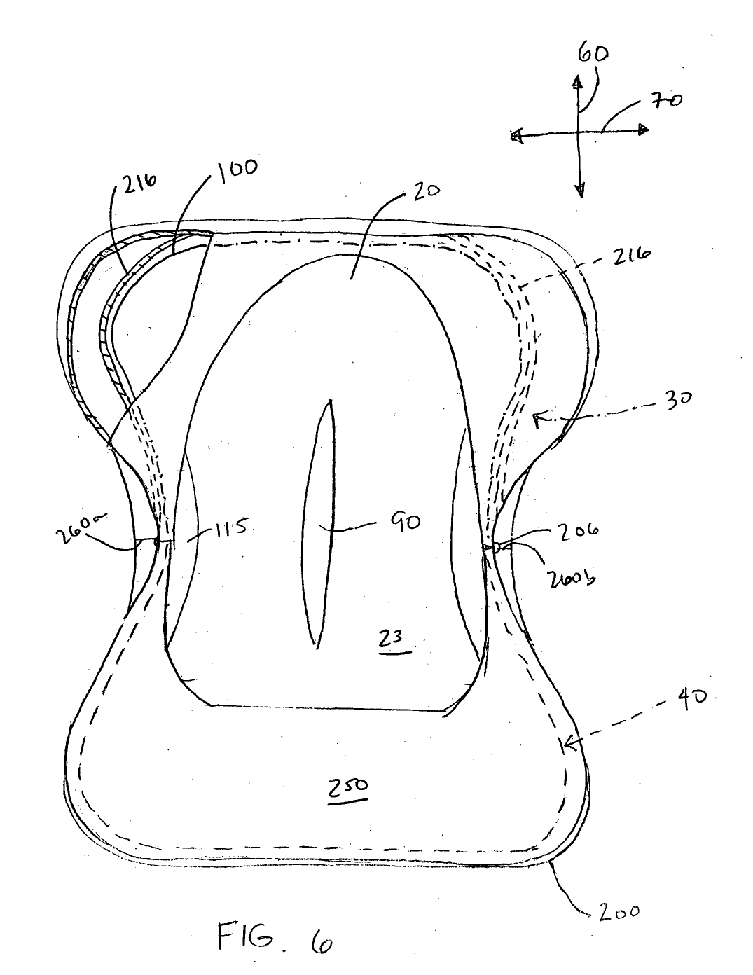

Referring now to FIG. 6, first shell member 202 is configured such that it can accommodate the rear portion 40 and part of the crotch portion 50 of absorbent garment 100. In particular, shell member 202 may be defined by a pan 210a, the pan defined by a floor 212a and outer side walls 214a. Likewise, second shell member 204 is configured such that it can accommodate the front portion 30 and part of the crotch portion 50 of absorbent garment 100. Shell member 204 may be defined by a pan 210b, the pan defined by a floor 212b and outer side walls 214b.

The outer edge of side wall 214 is defined by a rim. In particular, shell member 202 has a rim 218a, and shell member 204 has a rim 218b. The overall “footprint” defined by each rim 218 a,b may be substantially identical so that when the encasement 200 is in a closed state, the rims 218 a,b fit together in a complementary fashion along the rim. The rims may be flush as shown, or one or both rims 218 a,b may have a feature such as a tab or other extended member (not shown) to aid the user in opening encasement 200.

Shell members 202 and 204 may be prevented from shifting in opposite directions within the same plane when encasement 200 is in a closed state. For example, this shifting may be prevented by disposing a structural member, e.g. a lip, post, or other male-female type connection, from one shell member into the opposite shell member. As shown by way of example in FIG. 2, a lip 220 extends outwardly from rim 218b (or in the alternative, outwardly from rim 218a). Lip 220 may be continuous as shown, or discontinuous, and may engage the opposite rim 218 with a friction or slip fit. This connection prevents sideways movement of shell members 202 and 204, and if a friction fit, may keep the lid in a closed condition until an opening force is applied.

As described above, shell members 202 and 204 may have a substantially common footprint so that they fit together. However, the absorbent garment 100 may not be symmetric about a lateral axis 101. To improve the fit of garment 100 within encasement 200, an optional set of ribs 216 may be used. Ribs 216 may not be visible when the package 200 is in a closed position unless a transparent material is used to construct shell member 202. As can be seen in FIGS. 6 and 7, ribs 216 form an inner wall that generally follows the contour of at least a portion of garment 100, for example, the front portion 30 of garment 100.

Referring to FIG. 7, the ribs 216 may be integrally attached to the floor 212a of shell member 202. Each rib 216 may have an inner edge 230 that attaches to floor 212a, a first end 232 that may and attach to wall 214, and a second end 234 that may attach to wall 214a near hinge 206. Ribs 216 may extend normally from the inner surface 236 of floor 212a. In other embodiments, rib 216 may have a non-continuous surface, or may be a partial wall that does not attach to shell member 202 at ends 232, 234. For example, a series of post members (not shown) could operate to keep the garment 100 in place.

Encasement 200 may include a securement structure that functions to keep the article(s) 100 from inadvertently falling out of holding cavity 241. The securement structure or member may be a collar 240 as shown in FIGS. 2 and 6, or a set of spaced apart fingers, clips, bands, or the like. Collar 240 can be used to cover a portion of the cavity 241 to expose enough of garment 100 for selective dispensing.

An opening 243 is defined by collar 240. The opening 243 exposes an upper one of the absorbent garments 100 for dispensing without generally exposing the underlying absorbent articles. For example, opening 243 may be such that essentially only the topmost garment 100 is exposed by the opening 243, whether it be the garment-facing surface or body-facing surface of garment 100. Opening 243 has a size and shape sufficient to allow a user to grasp the exposed garment 100 and pull the article from the cavity 241 in the direction generally transverse to a plane of the opening 243.

Referring again to FIG. 7, collar 240 may have a two part construction, namely, a front collar portion 242 and a rear collar portion 244. Front collar portion 242 may be used to cover a portion of front waist region 30, and a rear collar portion 244 maybe used to cover a portion of back waist region 40. The collar's outer edge 246a may be substantially the same shape as corresponding rim 218a. Likewise, collar outer edge 246b may have the same shape as rim 218b, and may follow an inner edge of rim 218, substantially and continuously.

It is further contemplated that outer edges 246 a,b may contact corresponding walls 214 a,b at only select regions. For example, if collar 240 had a decorative element (e.g. a lacy pattern) it may not continuously follow rims 218 a,b. Inner edges 248 a,b together define the opening 243, the function of which is described above. Inner edges 248 a,b may have smooth, irregular or angular edges for a decorative effect. Inner edges 248 a,b may not be symmetric about hinge 206 as shown. In the alternative, the inner edges 248 a,b may be symmetric and form an oval, circle, square, rectangle, or any other symmetric design.

Each collar portion 242, 244 may be configured from a sheet of material and have the stiffness required to hold the garments 100 in place within cavity 241. Collar 240 may be constructed from a relatively stiff material, or may flex. For instance, when the user pulls the garment 100 from opening 243, the collar 240 may flex substantially to place less force in the garment 100 during removal.

Each collar portion 242, 244 may be integrally attached to walls 214 a and b respectively, for example, through injection molding. In the alternative, one or both collar portions 242, 244 may be selectively removable from shell members 202, 204, respectively. In an alternative embodiment, the each collar portion may have an opening therein for additional access to the cavity 241.

Collar 240 may include indicia that is informative, decorative or entertaining. In addition, collar 240 could be provided separately and snapped into place by the user. This would allow the user to choose from a variety of designs to customize the encasement. For instance, the collar 240 may be decorated with animals, fairytale and cartoon characters, photographs, different colors, provide inspirational messages, and the like. In addition, collars 240 may be scented, and the user could select from various scents.

Collar 240 may be positioned for use so that an exposed collar surface 250 is below the level of each rim 218 a,b. When encasement 200 is in a closed state, lip 222 may contact the surface 250 located in the opposite shell member. Referring to FIG. 3, should opposite forces be applied to each shell member 202, 204 in the directions 258 a and b respectively, the collar 240 can aid in preventing the shell member 204 from being pushed into shell member 202.

Hinge 206 may be integrally or non-integrally attached to both shell members 202 and 204 and configured to allow each shell member to rotate about hinge 206 at rear edges 260 a, b. For example, as is known by those skilled in the art, one or more sleeves (not shown) may extend from edges 260 a,b, and be subsequently aligned so that pins can slide into each sleeve to form a rotatable connection between shell members 202, 204. In an alternative embodiment, the shell members may be integrally connected by a relatively thin, flexible leaf member (not shown). However, one skilled in the art will realized that many types of hinges may be used to connect shell members 202 and 204.

A biasing structure may be included to operate in conjunction with hinge 206. Any type of resilient biasing device, including spring devices or a biased flexible leaf spring tape that could be used to join shell members 202, 204. In operation, the biasing structure may be incorporated with the hinge 206 to bias the encasement 200 into an open state. In this way, the encasement 200 will stay open and not tend to close as the garment 100 is being pulled from opening 243. Alternatively, the bias could operate to put the encasement 200 into a closed state.

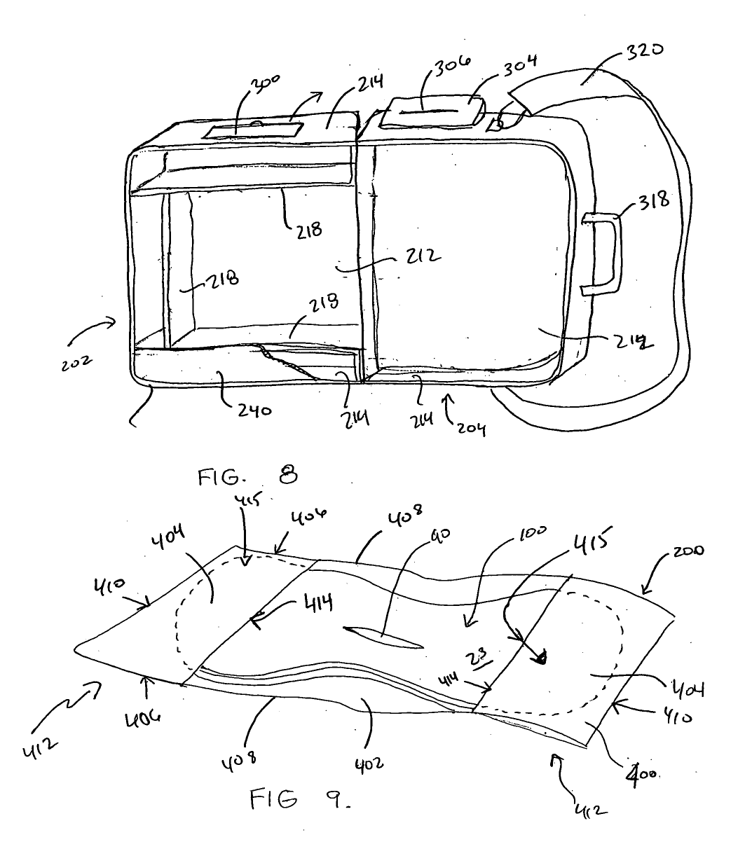

Referring to FIG. 8, accessories (not shown) may be stored in the encasement 200 along with garment 100. Accessories could include items such as wipes, tubes of ointment, containers of powder, changing pads, or the like. For example, accessories could be contained in any volume defined by encasement 200 that would not normally be occupied by stack 192. It is noted that “accessories” as used in the context of this invention is intended to broadly relate to the use of the absorbent articles, and may even include soiled diapers.

To prevent the accessories from freely moving about the cavity 241, the accessories could further be stored in a volume defined between an inner structure like rib 218 and outer wall 214, or an inner structure separate from the rib and outer wall. The accessories may be secured within such a space and accessed from port 300 located in the shell member 202 (or 204), perhaps in the floor 212 or outer wall 214. Port 300 allows access to accessories when encasement 200 is in a closed state. It is further contemplated that such accessories could be accessed when encasement 200 is in an open state. For example, the volume defined between rib 218 and wall 214 may be open, or covered by a collar 240. If covered by a collar 240, collar 240 may be made to be selectively removable or openable and/or may include a port (not shown).

In the alternative, or in addition thereto, the accessories may be stored in a volume that is exterior to the volume defined within encasement 200. For example, it is contemplated that a separate pod 304 could be selectively or permanently attached to an exterior portion of encasement 200. An opening 306 is provided for selective access to the accessories contained within pod 304. Pod 304 may be a flexible or non-flexible structure. The invention is not limited to the sizes and types of storage volumes depicted in the Figures.

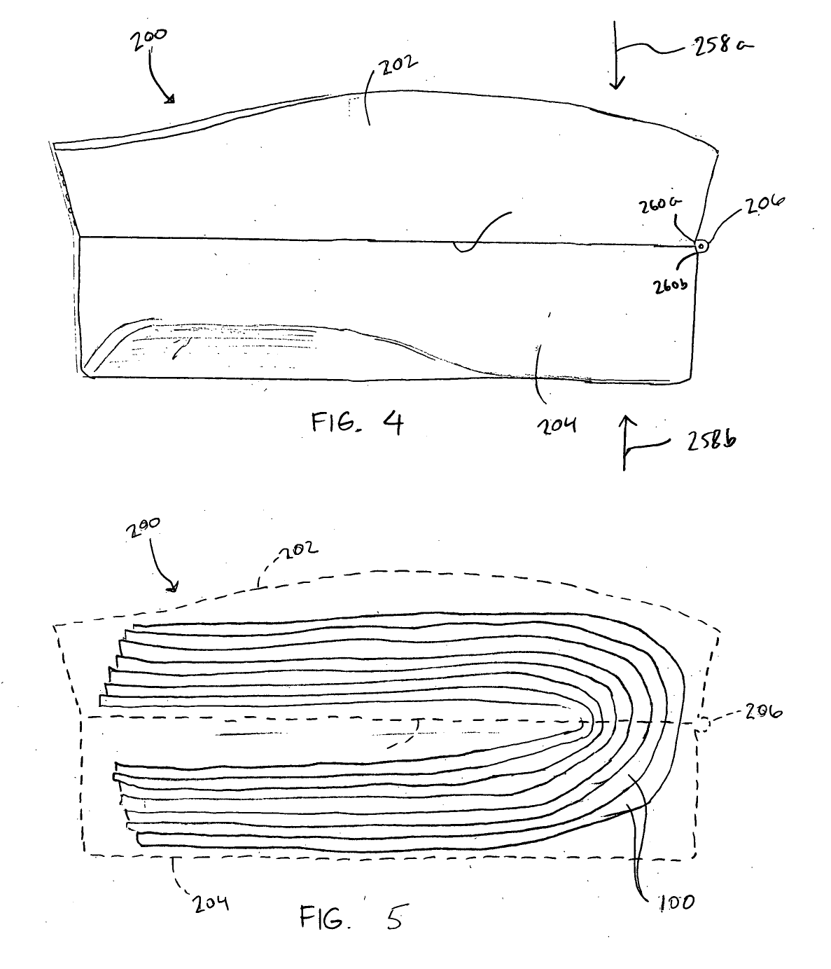

A viewing window 280 may be provided in the body 192 so that a user can ascertain the type and remaining number of garment 100 in the encasement 200 without opening the encasement. In this regard, the window 280 may be defined on one or both side walls 214 a,b, such that the number of articles in the stack 192 can be readily determined, as illustrated in FIG. 4. Preferably, the window 280 is located near to where the garments are folded, for example, nearer to where the hinge 206 is located. The garments may be thicker near the fold, and therefore, easier to count. The window 280 may be defined by an uncovered opening or an opening having a clear or translucent covering or panel.

Any conventional closing mechanism may be provided to secure the shell member 202 relative to the shell member 204. In the embodiment of FIGS. 3-8, the closing mechanism may be the fit between lip 222 and opposing rim 218. It is further contemplated that extending inwardly from the rim 218 or outwardly from lip 222 is a tab (not shown) that can be disposed in a detent located on the opposite shell member as is known in the art. For example a tab extending outwardly from lip 222 can engage a detent located on a wall 214 just below inner rim 218. It should be understood that this embodiment is illustrative only, and that number of mechanical or adhesive locking/fastening devices may be utilized to releasably secure the shell member 202 to the shell member 204 to prevent inadvertent opening of encasement 200. For example, the there could be a wrap-like member extending from the encasement; the wrap-like member may include an opening/closing feature, and/or a storage volume for accessories.

In an alternative embodiment, the fit that serves to keep the encasement closed may be enhanced or replaced by a material or other structure that provides a higher coefficient of friction with respect to the wall 214. In yet other embodiments, tapes, hook and loop fasteners, magnets, bands, clasps, clips, snaps, buckles, or the like may be used. Further, lip 222 may be omitted and/or a biasing member may be used to keep encasement 200 in a closed position.

In the illustrated embodiment of FIGS. 2 and 3, a textured portion 310 may be provided by one or more boss members 312 that extend outwardly from one or both walls 214 a, b. Texture portion 310 can serve to aid the user in opening the encasement 200 by indicating a specific region for the user to apply a pushing force to open encasement 200. In addition, the textured portion may locally increase the coefficient of friction to wall(s) 214.

To further assist with opening of encasement 200, one or both walls 214 a, b may be slanted inwardly from the floor to the rim, especially in the front region 251 of encasement 200. This enables the user to more easily apply pressure to each shell member 202, 204 for opening encasement 200.

A handle and/or a carrying strap may be provided for encasement 200. As seen by way of example in FIG. 8, a handle 318 may be attached to an exterior portion of encasement 200, such as on a wall 214. Handle 318 may be separately or integral with the encasement, perhaps by injection molding. Instead of or in addition thereto, a strap 320 may also be attached to an exterior portion of encasement 200, such as on a wall 214.

In operation, for the encasement embodiment illustrated in FIGS. 3-8, the shell member 202 may be opened by simply rotating the shell member 202 and/or shell member 204 about hinge 206. To fill or refill the encasement 200, a stack 192 of absorbent articles are placed within cavity 241. The stack is folded by closing the encasement 200. To access a fresh absorbent article, the encasement 200 is opened and the top article is pulled away from the remainder of stack 192. If a collar 240 is present, the top article is pulled through opening 243. In one embodiment, the article is garment 100 and it is unnecessary to unfold tabs or ears are after removing it from the encasement 200.

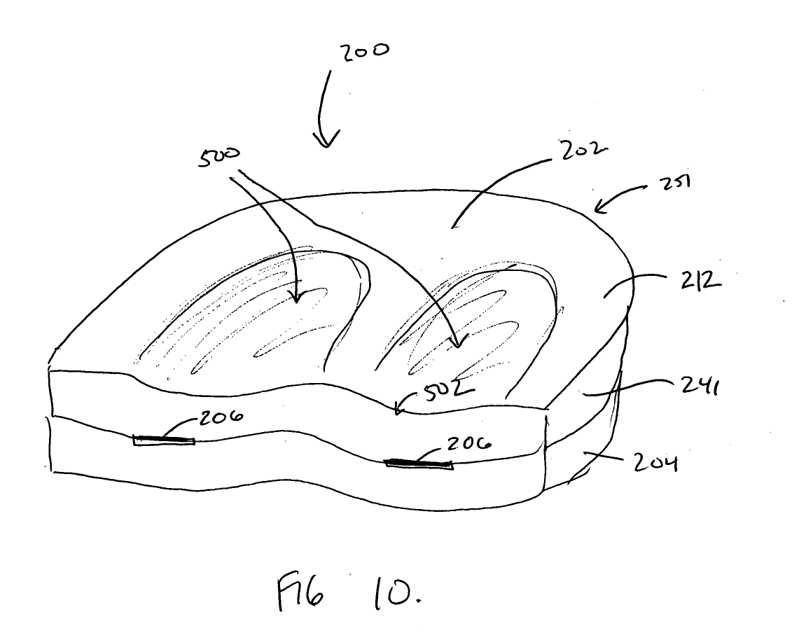

In an alternative embodiment, the encasement 200 may include a folder-style body 400. Referring to FIG. 9, the folder-style body 400 may be a flexible structure having a rectangular body 402 with a pair of panels 404 attached to a common surface at opposite ends thereof. The panels 404 may also be rectangular in shape and have two opposite side edges 406 sealed to the body on or near body side edges 408. The outermost edge 410 of panel 404 maybe formed with a seal or by folding the body over at each respective end region 412. Panel inner edge 414 is left unattached to body 402. Pockets 415 are defined between each panel 402 and body 404. A single article or stack 192 of absorbent articles, e.g. garments 100, can be selectively held in the body 400 by pockets 415. Garments 100 may be placed into the pockets 415 so that either the garment-facing surface or the body facing surface 23 is exposed. The body 402 and panels 404 are not limited in scope to a rectangular shape.

In yet another embodiment, shown in FIG. 10, the encasement is configured so that it may be used as a booster seat. In one of the shell members, e.g. shell member 202, a pair of concave portions 500 may be provided so as to conform the posterior side of a user when seated. The concave portions may lead up to edge 502 to prevent undue pressure on the back legs of the user. A vast number of booster seat configurations are within the spirit and scope of this embodiment.

A suitable method for producing encasement 200 of FIGS. 3-8 is injection molding or any other suitable technique familiar to those of ordinary skill in the art. One non-limiting suitable material of construction for the encasement 200 includes a plastic such as polypropylene. Other suitable materials may include paper, metal, thermoplastic elastomers, or composite materials. Likewise, a suitable method for producing encasement 200 of FIG. 9 is extrusion or any other suitable technique familiar to those of ordinary skill in the art. One non-limiting suitable material of construction for the encasement 200 includes a non-woven fabric. Other suitable materials may include plastic sheeting; paper; metal, vinyl; knits, non-woven, and woven fabrics, or the like.

It should be appreciated by those skilled in the art that various modifications and variations can be made to the embodiments of the absorbent article described herein without departing from the scope and spirit of the invention as set forth in the appended claims and equivalents thereof. For example, as mentioned, the absorbent articles may be feminine pads or the like. Feminine or adult care accessories could include wipes, deodorant spray, topical medication, or the like.

1. A parcel of disposable personal-care products designed to be worn on the lower torso, the parcel comprising:
a first product comprising a pair of first surfaces, a first body-facing surface and a first garment-facing surface; and a first fold-line;
a second product comprising a pair of second surfaces, a second body-facing surface and a second garment-facing surface; and a second fold-line;
a stack comprising the first product and the second product wherein one of the pair of first surfaces is in contacting relationship with one of the pair of second surfaces; and
an encasement adapted to have an open condition and a closed condition; wherein the stack is disposed within the encasement;
whereby when the encasement is in a closed condition, the first product is substantially folded at the first fold line and the second product is substantially folded at the second fold line, and when the encasement is in an open condition, the first and second products are at least partially unfolded.

2. The parcel of claim 1 wherein the first fold-line and the second fold-line are substantially aligned when the stack is in an unfolded state.

3. The parcel of claim 1 wherein the encasement comprises:
a first shell member;
a second shell member mateable with the first shell member to define an inner recess to enclose the stack; and
a hinge that attaches the first shell member to the second shell member.

4. The parcel of claim 3 wherein the first product and the second product are diapers.

5. The parcel of claim 3 wherein a lid member comprises an inner surface and an outer surface, and an accessory holder disposed on the outer surface.

6. The parcel of claim 3 further comprising a stack holder adapted to dispense the first and second products from the encasement.

7. The parcel of claim 6 wherein the stack holder comprises a collar.

8. The parcel of claim 7 wherein the collar is scented.

9. The parcel of claim 6 wherein the stack holder is adapted to cover about 4 to about 90 percent of one of the first pair of surfaces of the first product.

10. The parcel of claim 1 wherein the encasement comprises a folder-style body.

11. The parcel of claim 1 wherein the encasement comprises a bacterial resistant material.

12. The parcel of claim 1 wherein the encasement is biased to the open state, and further comprises a fastening assembly for selectively maintaining the encasement in the closed state.

13. An encasement for selectively dispensing absorbent products for wear about the lower torso, the encasement comprising:
a first shell member;
a second shell member mateable with the first shell member to define an inner recess adapted to enclose a stack of absorbent articles;
a stack holder adapted to dispense the absorbent articles; and
a connector that attaches the first shell member to the second shell member so that the encasement may be selectively opened and closed.

14. The encasement of claim 13 wherein the stack holder comprises a collar.

15. The encasement of claim 13 wherein the encasement includes recesses on the first shell member, the recesses adapted to fit the posterior side of a seated person for use as a booster seat.

16. The encasement of claim 13 wherein one of the first shell member or the second shell member has an accessory holder disposed on an exterior surface thereof.

17. A parcel comprising:
a plurality of disposable diapers, wherein the plurality of disposable diapers comprise a stack of unfolded diapers;
an encasement adapted to have an open condition and a closed condition;
wherein the stack is disposed within the encasement so that when the encasement is in a closed condition, the stack is substantially folded, and when the encasement is in an open condition, the stack is at least partially unfolded.

18. The parcel of claim 17 wherein the encasement further comprises an accessory holder for containing accessories.

19. The parcel of claim 17 wherein encasement comprises a clam-shell type package.

20. The parcel of claim 17 wherein the encasement further comprises a window for viewing the number of disposable diapers contained therein.
